(19) United States
(12) Patent Application Publication
(10) Pub. No.: US 2022/0107880 A1
(43) Pub. Date: Apr. 7, 2022

(54) SYSTEM AND METHOD FOR PRESENTING A MONITORING DEVICE IDENTIFICATION

(71) Applicant: KPN INNOVATIONS, LLC., Lakewood, CO (US)

(72) Inventor: Kenneth Neumann, Lakewood, CO (US)

(21) Appl. No.: 17/062,710

(22) Filed: Oct. 5, 2020

(51) Int. Cl. G06F 11/34; G06N 20/00; G06F 11/30; G16H 50/20; G16H 10/20

(52) U.S. Cl. CPC G06F 11/3495; G06N 20/00; G16H 10/20; G16H 50/20; G06F 11/302

(57) ABSTRACT

A system for presenting a monitoring device identification includes a computing device configured to obtain a user profile from a graphical user interface, identify a user condition as a function of the user profile, determine a monitoring device of a plurality of monitoring devices as a function of the user condition, wherein determining further comprises, obtaining a monitor training set, wherein the monitor training set relates a condition element to a detection method and determining the monitoring device as a function of a monitoring machine-learning process and the user condition, wherein the monitoring machine learning process is configured as a function of the monitoring training set; and present the monitoring device at the graphical user interface.

### FIELD OF THE INVENTION

The present invention generally relates to the field of artificial intelligence. In particular, the present invention is directed to a system and method for presenting a monitoring device identification.

### BACKGROUND

Monitoring devices are flooding the consumer market every day, which is confusing individuals as to which device to purchase. This is further complicated by the lack of corporations disclosing all of the benefits a monitoring device may be capable of. The lack of transparency in the consumer market from corporations manufacturing monitoring devices has resulted in numerous frustrated individuals that fail to receive the entirety of benefits that could be received.

### SUMMARY OF THE DISCLOSURE

In an aspect, a system of presenting a monitoring device identification includes a computing device, the computing device further configured to obtain a user profile from a graphical user interface, identify a user condition as a function of the user profile, wherein identifying further comprises acquiring a condition training set relating at least a user profile to a condition and using a condition machine-learning process, wherein the condition machine learning process is configured using the condition training set, determine a monitoring device of a plurality of monitoring devices as a function of the user condition, wherein determining further comprises, obtaining a monitor training set, wherein the monitor training set relates a condition element to a detection method and determining the monitoring device as a function of a monitoring machine-learning process and the user condition, wherein the monitoring machine learning process is configured as a function of the monitoring training set; and present the monitoring device at the graphical user interface.

In another aspect, a method of presenting a monitoring device identification includes obtaining, by a computing device, a user profile from a graphical user interface, identifying, by the computing device, a user condition as a function of the user profile, wherein identifying further comprises acquiring a condition training set relating at least a user profile to a condition and using a condition machine-learning process, wherein the condition machine learning process is configured using the condition training set, determining, by a computing device, a monitoring device of a plurality of monitoring devices as a function of the user condition, wherein determining further comprises, obtaining a monitor training set, wherein the monitor training set relates a condition element to a detection method and determining the monitoring device as a function of a monitoring machine-learning process and the user condition, wherein the monitoring machine learning process is configured as a function of the monitoring training set; and presenting, by the computing device, the monitoring device at the graphical user interface.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of a system for presenting a monitoring device identification;

FIG. 2 is a block diagram of an exemplary embodiment of a machine-learning module;

FIG. 3 is a block diagram of an exemplary embodiment of a user preference obtained by a graphical user interface;

FIG. 4 is a block diagram of an exemplary embodiment of a device database;

FIG. 5 is a process flow diagram illustrating an exemplary embodiment of a method of presenting a monitoring device identification; and

FIG. 6 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for presenting a monitoring device identification. In an embodiment, this system presents a monitoring device identification as a function of a user condition and a user preference. Aspects of the present disclosure can be used to present a monitoring device that at least monitors one or more elements relating to a user condition. This is so at least in part, because the system obtains a user profile from the user and determines, via a machine-learning process, the monitoring device that is capable of detecting and monitoring the user condition. Exemplary embodiments illustrating aspects of the present disclosure are described below in the context of several specific examples.

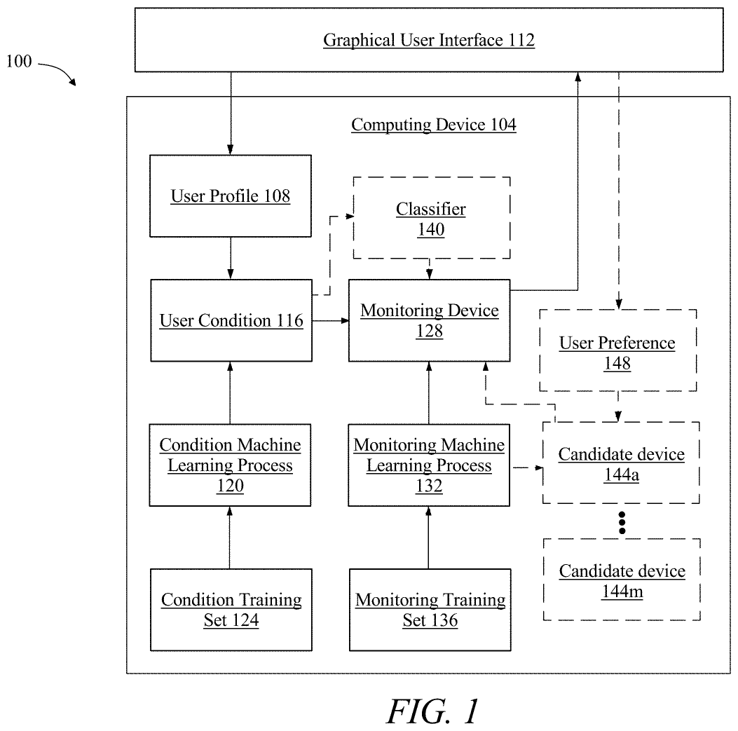

Referring now to FIG. 1, an exemplary embodiment of a system 100 for presenting a monitoring device identification is illustrated. System 100 includes a computing device 104. Computing device 104 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Computing device 104 may include a single computing device operating independently, or may include two or more computing device operating in concert, in parallel, sequentially or the like; two or more computing devices may be included together in a single computing device or in two or more computing devices. Computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device 104 may include but is not limited to, for example, a computing device or cluster of computing devices in a first location and a second computing device or cluster of computing devices in a second location. Computing device 104 may include one or more computing devices dedicated to data storage, security, distribution of traffic for load balancing, and the like. Computing device 104 may distribute one or more computing tasks as described below across a plurality of computing devices of computing device, which may operate in parallel, in series, redundantly, or in any other manner used for distribution of tasks or memory between computing devices. Computing device 104 may be implemented using a “shared nothing” architecture in which data is cached at the worker, in an embodiment, this may enable scalability of system 100 and/or computing device.

Computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, computing device 104 is configured to obtain a user profile 108. As used in this disclosure “user profile” is a set of data having characteristics unique to a user. A user profile 108 may include a questionnaire, survey, and/or biological extraction that is specific to a particular user. As used in this disclosure a “questionnaire” is a written set of questions of a plurality of written questions that may indicate one or more characteristics, elements, or traits associated with user profile 108. For example, and without limitation, a questionnaire may include providing a user with a written and/or digital form in which the user has to answer about any symptoms, signs, or condition impacts they may be experiencing. A further non-limiting example a questionnaire may include questions such as “are you experiencing chest pains?” or “are you experiencing blurred vision?” or “are you experiencing shortness of breath?”. A “biological extraction”, as used in this disclosure includes at least an element of user biological data. As used in this disclosure, “biological data” is data indicative of a person's biological state; biological state may be evaluated with regard to one or more measures of health of a person's body, one or more systems within a person's body such as a integumentary system, a skeletal system, a muscular system, a nervous system, a endocrine system, a cardiovascular system, a urinary system, a respiratory system, a lymphatic system, a digestive system, a reproductive system, or the like, one or more organs within a person's body, and/or any other subdivision of a person's body useful for diagnostic or prognostic purposes. For instance, and without limitation, a particular set of biomarkers, test results, and/or biochemical information may be recognized in a given medical field as useful for identifying various disease conditions or prognoses within a relevant field. As a non-limiting example, and without limitation, biological data describing red blood cells, such as red blood cell count, hemoglobin levels, hematocrit, mean corpuscular volume, mean corpuscular hemoglobin, and/or mean corpuscular hemoglobin concentration may be recognized as useful for identifying various conditions such as dehydration, high testosterone, nutrient deficiencies, kidney dysfunction, chronic inflammation, anemia, and/or blood loss. Biological extraction data may alternatively or additionally include any data used as a biological extraction as described in U.S. Nonprovisional application Ser. No. 16/502,835, filed on Jul. 3, 2019, and entitled “METHODS AND SYSTEMS FOR ACHIEVING VIBRANT CONSTITUTION BASED ON USER INPUTS,” the entirety of which is incorporated herein by reference.

Continuing to refer to FIG. 1, computing device 104 is configured to obtain user profile 108 from a graphical user interface 112. As used in this disclosure “graphical user interface” is a form or other graphical element having data entry fields, where a user may select one or more fields to enter one or more elements rating to user profile 108. Graphical user interface 112 may provide a drop-down menu and display one or more user profile elements where a user may select one or more elements relating to user profile 108. Graphical user interface 112 may list one or more categories relating to user profile 108, such as location of pain in relation to a human body, level of pain experiences, or conditions associated with user medical lineage. For example, and without limitation, a user may have primary congenital glaucoma, a genetic condition that is inherited in an autosomal dominant pattern, wherein graphical user interface 112 may display this category for a user to select as a function of the user medical lineage. Graphical user interface 112 may list one or more sub-categories of user profile 108 relating to additional signs, symptoms, or condition impacts associated with previous user medical history. For example, graphical user interface 112 may display a category associated with rhabdomyolysis such as kidney complications or muscular dystrophy.

Still referring to FIG. 1, computing device 104 is configured to identify a user condition 116 as a function of user profile 108. As used in this disclosure “user condition” is a list or collection of current or potential ailments and/or diseases, and/or precursor states to such ailments and/or diseases, including but not limited to physical, spiritual, and/or psychological ailments and/or diseases correlating to any resulting impact on the user. For example, a physical user condition may include, without limitation, Influenza, Rhinovirus, Obesity, COVID-19, EEE, CRE, Ebola, Enterovirus D68, Influenza, Hantavirus, Hepatitis A, Hepatitis A, HIV/AIDS, Diabetes (Type I or Type II), Multiple Sclerosis, Chron's Disease, Colitis, Lupus, Rheumatoid Arthritis, Allergies, Asthma, Relapsing Polychondritis, Scleroderma, Liver Disease, Heart Disease, Cancer, and the like thereof. For example, a spiritual user condition may include, without limitation, religious conflicts, chakra blockages, existential crisis, or the like thereof. For example a psychological user condition may include, without limitation, Alzheimer's, Parkinson's, alcohol or substance abuse disorder, anxiety disorder, ADD, ADHD, bipolar disorder, depression, eating disorder, obsessive-compulsive disorder, opioid use disorder, PTSD, schizophrenia, depersonalization disorder, dissociative amnesia and/or fatigue, anorexia, bulimia, sleep disorders, wake disorders, paraphilic disorders, sexual disorders, child mental disorders, personality disorders, gender dysphoria, depression, and the like thereof. Persons skilled in the art, upon reviewing the entirety of this disclosure, will be aware of various alternative or additional user conditions that may be used consistently with this disclosure.

With continued reference to FIG. 1, computing device 104 may identify user condition 116 as a function of a condition machine-learning process 120. As used in this disclosure “condition machine-learning process 120” is a machine learning process that automatedly uses training data and/or a training set to generate an algorithm that will be performed by a computing device 104 and/or module to produce outputs given data provided as inputs; this is in contrast to a non-machine learning software program where the commands to be executed are determined in advance by a user and written in a programming language. Condition machine learning process 120 may consist of any supervised, unsupervised, or reinforcement machine-learning process that computing system 104 may or may not use in the determination of the user condition. Condition machine-learning process 120 may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, markov decision processes, Deep Deterministic Policy Gradient (DDPG), or the like thereof. Condition machine learning process 120 may be generated as a function of a condition training set 124. As used in this disclosure “condition training set” is a training set that correlates a questionnaires, surveys, and/or biological extractions to a condition that a user may be experiencing. Condition training set 124 may include, and without limitation, the presence of the biomarker chitinase 3-like 1 (cartilage glycoprotein-39) (CHI3L1), C reactive protein, pentraxin-related (CRP), epidermal growth factor (beta-urogastrone) (EGF), interleukin 6 (interferon, beta 2) (IL6); leptin (LEP); matrix metallopeptidase 1 (interstitial collagenase) (MMP1); matrix metallopeptidase 3 (stromelysin 1, progelatinase) (MMP3); resistin (RETN); serum amyloid A1 (SAA1); tumor necrosis factor receptor superfamily, member 1A (TNFRSF1A); vascular cell adhesion molecule 1 (VCAM1), and, vascular endothelial growth factor A (VEGFA), which may correlate to the condition of rheumatoid arthritis in a user. As a further non-limiting example condition training set 124 may include the presence of signs, symptoms, or condition impacts associated with feeling nervous, restless, having a sense of impending danger, increased heart rate, hyperventilation, sweating, trembling, feeling weak or tired, insomnia, gastrointestinal problems, avoidance of potential anxiety triggers, or the like thereof, which may correlate to the condition of an anxiety disorder.

Still referring to FIG. 1, computing device 104 is configured to determine, as a function of user condition 116, a monitoring device 128 of a plurality of monitoring devices. As used in this disclosure a “monitoring device” is a smart electronic device that is worn close to and/or on the surface of the skin, wherein the device can detect, analyze, and transmit information concerning a body signal such as a vital sign, and/or ambient datum, wherein allowing immediate biofeedback to be sent to the user wearing the device. Monitoring device 128 my consist of, without limitation, near-body electronics, on-body electronics, in-body electronics, electronic textiles, smart watches, smart glasses, smart clothing, fitness trackers, body sensors, wearable cameras, head-mounted displays, body worn cameras, Bluetooth headsets, wristbands, smart garments, chest straps, sports watches, fitness monitors, and the like thereof. Monitoring device 128 may include, without limitation, earphones, earbuds, headsets, bras, suits, jackets, trousers, shirts, pants, socks, bracelets, necklaces, brooches, rings, jewelry, AR HMDs, VR HMDs, exoskeletons, location trackers, and gesture control wearables. Additionally or alternatively, monitoring device 128 may include, without limitation, any device that further collects, stores, and analyzes data associated with heart rate, calories burned, steps walked, blood pressure, release of biochemicals, time spent exercising, seizures physical strain, or the like thereof. Monitoring device 128 may include, without limitation, any device that obtains information relating to forecasting changes in mood, stress, and health, measuring blood alcohol content, athletic performance, monitoring how sick the user is, health risk assessment applications, long-term monitoring of patients, short-term monitoring of patients, automatic documentation of care activities, of the like thereof.

Still referring to FIG. 1, computing device 104 may determine monitoring device 128 by measuring situational information. As used in this disclosure “situational information” is information relating the location of an at least a first condition element to the location of an at least second condition element. As used in this disclosure a “condition element” is a quality, component, and/or integrant of a condition. For example, and without limitation, a condition element may include a cough, respiratory distress, decreased O₂ saturation level, cognitive dysfunction, death, lethargy, cognitive dysfunction, aches and pains, and sinus pressure for the condition of influenza. Situational information may relate two or more condition elements that relate to one or more monitoring devices. For example and without limitation, a first monitoring device may provide a first condition element at the location of a wrist, while a second monitoring device may provide a second condition element at the location of the waist. The situational information would then relate to the two separate locations in which the two or more condition elements are collected. Additionally or alternatively, one or more monitoring devices may relate to two or more condition elements that relate to separate locations of the human body. For example, with without limitation, a single monitoring device may relate a first condition element of sleep function as well as a second condition element of perspiration rate. As a further non-limiting example an implantable monitoring device may relate one or more condition elements such as heart rate, distance walked, body temperature, oxygen saturation, breathing rate, and the like thereof from the single monitoring device. Situational information may relate the one or more monitoring devices to the at least two condition elements to a single user condition, wherein a single user condition is monitored according to the two or more condition elements. For example and without limitation, two condition elements monitored of breathing rate and blood pressure may be necessary to monitor the user condition of anxiety, wherein the two condition elements are monitored by two or more monitoring devices located at two or more locations of the body. As a further non-limiting example, two condition elements of O₂ saturation and lethargy may be necessary to monitor the user condition of COVID-19, wherein the two condition elements are located in separate locations of the body which are monitored using a single monitoring device.

Still referring to FIG. 1, computing device 104 is configured to determine monitoring device 128 as a function of a monitoring machine-learning process 132. As used in this disclosure “monitoring machine-learning process” is a machine learning process that automatedly uses training data and/or a training set to generate an algorithm that will be performed by a computing device and/or module to produce outputs given data provided as inputs; this is in contrast to a non-machine learning software program where the commands to be executed are determined in advance by a user and written in a programming language. Monitoring machine learning process 132 may consist of any supervised, unsupervised, or reinforcement machine-learning process that computing system 104 may or may not use in the determination of the user condition. Monitoring machine-learning process 132 may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, monitoring machine learning process 132 is generated as a function of a monitoring training set 136. As used in this disclosure “monitoring training set” is a training set that correlates a condition element to a detection method, wherein a condition element is described in detail above. As used in this disclosure “detection method” is a route, technique, and/or strategy that an electronic device uses for the monitoring of the biological state of an individual, wherein a biological state, which is described above, may include any physical status of an individual such as height, weight, blood pressure, pulse, vision, heart function, lung function, gastrointestinal function, auditory function, olfactory function, lingual function, posture, joint function, muscular strength, flexibility, or the like thereof. Detection method be comprised of any method relating to a condition state of the user. As used in this disclosure a “condition state” is a metric or other quantitative value associating the user condition to the progression of the condition. For example, a condition state may be a value of 5 when a user has a fever due to influenza, while a condition state may be a value of 20 when a user has pneumonia and a fever due to influenza. Additionally or alternatively, monitoring machine learning process 132 may be generated as a function of a classifier 140

Further referring to FIG. 1, as used in this disclosure a “classifier” is a machine-learning model, such as a mathematical model, neural net, or program generated by a machine learning algorithm known as a “classification algorithm,” as described below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Classifier 140 may receive a monitoring device of a plurality of monitoring devices relating to a user condition, wherein the classifier may output one or more monitoring devices that at least detect one or more elements of the user condition. Computing device 104 and/or another device may generate a classifier using a classification algorithm, defined as a processes whereby a computing device 104 derives a classifier from training data. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. For example, and without limitation, a classifier 140 May receive a user condition input of anxiety, wherein a monitoring device comprising a heart rate detection method, a blood pressure detection method, and a fitness monitor may be outputted. As a further non-limiting example, classifier 140 may receive a user condition input of ischemic heart disease, wherein a monitoring device comprising a heart rate detection method, blood pressure detection method, and a fitness monitor, may be outputted for one monitoring device as well as a secondary monitoring device may be outputted relating to an electrocardiogram detection method.

Still referring to FIG. 1, computing device 104 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 104 may then calculate a likelihood table by calculating probabilities of different data entries and classification labels. Computing device 104 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 1, computing device 104 may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in a database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 1, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute I as derived using a Pythagorean norm: l=\sqrt{Σᵢ₌₀ⁿaᵢ²}, where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

Still referring to FIG. 1, monitoring machine-learning process 132 may be configured to determine a device score. As used in the disclosure a “device score” is a quantitative enumeration that characterizes a set value associated with a monitoring device in relation to the user condition, wherein the quantitative enumeration represents an ability to provide data concerning the condition. For example, and without limitation, a monitoring device that measures the quantity of fat a user has that includes detection methods of skinfold calipers, body circumference measurements, dual-energy X-ray absorptiometry, hydrostatic weighing, air displacement plethysmography, bioelectrical impedance analysis, bioimpedance spectroscopy, electrical impedance myograph, 3-D scanners, and multi-compartment models may receive a device score of 100, whilst a monitoring device that has detection methods of only bioelectrical impedance analysis and bioimpedance spectroscopy may receive a device score of 20. Device scores may be ranked; for instance, computing device 104 may select the highest-ranking device score, a certain number of highest-ranking device scores, or a certain number of device scores that fulfill a pre-configured threshold. Device scores may be compared to a preconfigured threshold, wherein a preconfigured threshold is a quantitative and/or numerical value for which the larger score triggers a selection. Computing device 104 may combine device scores that fail the pre-configured threshold, eliminating those device scores and ranking the remaining device scores. Additionally or alternatively, device score may alter as a function of the user condition. A monitoring device, which has a device score of 100 for a user condition of diabetes, may only have a device score of 10 for a secondary user condition such as eczema. Device score calculations may be generated as a function of a supervised machine-learning process, a neural net, a distance metric in relation to a classifier, and/or the like thereof. For example a linear regression technique may be used to train and/or select coefficients of a linear equation of data concerning a device score of a monitoring device and outputs the equation as a device score.

Still referring to FIG. 1, monitoring machine-learning process 132 may be configured to output a candidate monitoring device 144a of a plurality of candidate monitoring devices 144a-m. As used in this disclosure a “candidate monitoring device” is a monitoring device that is generated as a function of the monitoring machine-learning process, such that a detection method exists to monitor a user condition. Selection of candidate monitoring device 144 may be done using a threshold comparison to a stored quantitative value representing a minimal ranking for selection, ranking, significant positions in multiple alignment, profile construction, or the like thereof. For example, and without limitation, candidate monitoring device 144a-m may include a Fitbit, Apple watch, and/or Galaxy watch for the user condition of heart arrythmia to track the heart rate of a user. Computing device 104 may be configured to select the monitoring device from the plurality of candidate monitoring devices by displaying the plurality of candidate monitoring devices on the computing device. For example, computing device may display three monitoring devices associated with a user condition of depression, wherein monitoring devices include a Cervella cranial electrotherapy stimulator, a LivaNova VNS Therapy System, and a Flow Brain Stimulation Headset. Computing device 104 may then receive a user preference 148 ranking the plurality of candidate monitoring devices. As used in this disclosure “user preference” is a soft requirement provided by the users, in addition to the user profile, that reflects a wish, desire, want, and/or urge regarding an element of the monitoring device, wherein elements of the monitoring device are discussed below in detail and may include, without limitation, style, color, brand, software, price, availability, and the like thereof. For example, and without limitation, a user may be presented with a list of five smart watches, including an Apple watch, a Galaxy watch, a Fitbit Sense, a Fossil Gen 5, Tag Heuer Connected, and a Garmin Instinct, wherein a user preference may indicate that a user prefers a black wearable device that is in the $200.00-$299.99 price range, with a twist style, that is compatible with the user's smartphone, is a Fitbit brand, and is available to purchase today. Computing device 104 may then receive user preference 148 and select the Fitbit smartwatch as a function of the user preference.

With continued reference to FIG. 1, computing device 104 may perform machine-learning algorithms using a loss function analysis to select each monitoring device 128 from plurality of monitoring devices. In an embodiment, computing device 104 may compare one or more user preferences to a mathematical expression representing a plurality of user conditions. Mathematical expression may include a linear combination of variables, weighted by coefficients representing relative importance of each goal parameter. For instance, a variable such as user condition severity may be multiplied by a first coefficient representing the user condition status, a second user input such as total cost may be multiplied by a second coefficient representing the importance of cost, a degree of variance from and/or classified beneficial monitoring device may be represented as another parameter, which may be multiplied by another coefficient representing the importance of that parameter, a degree of variance from a preference for brand may be multiplied by an additional coefficient representing an importance of that parameter, or the like; persons skilled in the art, upon reviewing the entirety of this disclosure, will be aware of different variables that may be weighted by various coefficients. Use of a linear combination is provided only as an illustrative example; other mathematical expressions may alternatively or additionally be used, including without limitation higher-order polynomial expressions or the like.

With continued reference to FIG. 1, computing device 104 may generate a loss function algorithm utilizing the ranked plurality of candidate monitoring devices and a condition parameter. As used in this disclosure, a “loss function” is an expression an output of which an optimization algorithm minimizes to generate an optimal result. As used in this disclosure “condition parameter” is a measurable factor relating to the efficiency of monitoring a user condition. A condition parameter may include, an efficiency measurable factor, wherein an efficiency measurable factor relates to how effective a sensor of the wearable device may be at detecting human physiological statuses, wherein human physiological statuses may include heartbeat, blood pressure, body temperature, electrocardiograms, arrhythmias, cancerous indicators, body fat composition, or the like thereof. A condition parameter may be determined using one or more pressure sensors, humidity sensors, position sensors, piezo film sensors, force sensors, temperature sensors, optical sensors, or the like thereof. Condition parameters may vary as a function of the plurality of user conditions. For example a condition parameter for monitoring the heart beat using a pressure sensor may result in a value of 10, while a condition parameter for monitoring a heartbeat using a piezo electric sensor may result in a value of 20, wherein the piezo electric sensor would have a better efficiency of monitoring a heartbeat. Computing device 104 may then calculate a difference between the plurality of candidate monitoring devices and the condition parameters of a plurality of condition parameters associated with a monitoring device as a function minimizing the loss function. As a non-limiting example, computing device 104 may assign variables relating to a set of parameters, which may correspond to condition parameters as described above, calculate an output of mathematical expression using the variables, and select a monitoring device that produces an output having the lowest size, according to a given definition of “size,” of the set of outputs representing each of plurality of candidate monitoring devices; size may, for instance, included absolute value, numerical size, or the like. Computing device 104 may then select a monitoring device of a plurality of monitoring devices for the user as a function of the calculated difference.

Still referring to FIG. 1, computing device 104 is configured to present the monitoring device at graphical user interface 112. Computing device may present monitoring device 128 at graphical user interface, such that the monitoring device fulfills the user preference requirements as well as the user condition requirements. Computing device 104 may present one or more monitoring devices such that the user condition may be monitored as required. For example, and without limitation, one monitoring device may be presented for the monitoring of the user condition associated with heart arrythmias, through the detection method of heart rate. As a further non-limiting example, multiple monitoring devices may be presented for the monitoring of a user condition associated with COVID-19, such as a wearable fabric to monitor the bodies O₂ saturation level, a smart watch to monitor the heart rate, and/or a chest monitoring device that is capable of monitoring the respiratory rate of a used.

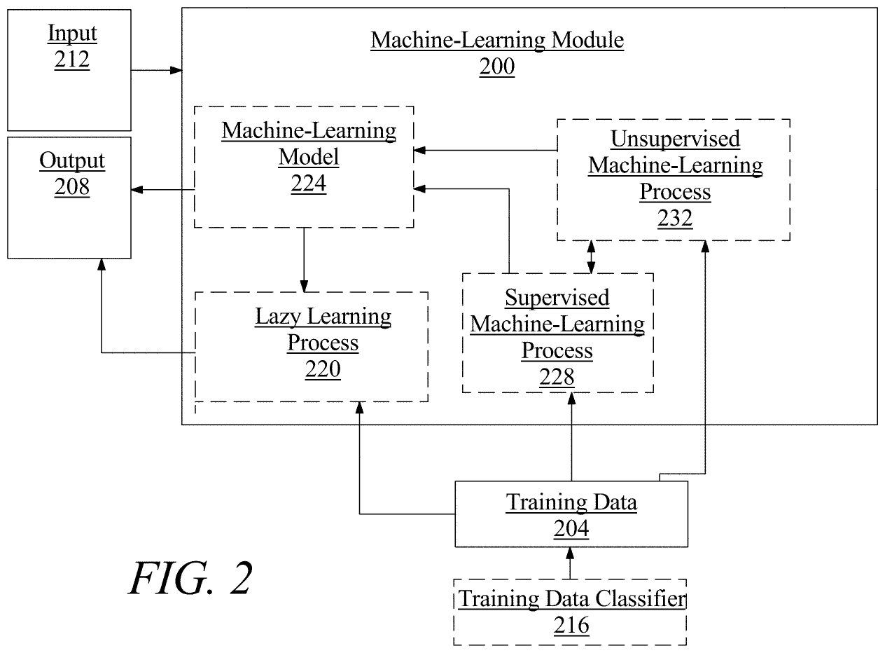

Referring now to FIG. 2, an exemplary embodiment of a machine-learning module 200 that may perform one or more machine-learning processes as described in this disclosure is illustrated. Machine-learning module may perform determinations, classification, and/or analysis steps, methods, processes, or the like as described in this disclosure using machine-learning processes. A “machine-learning process,” as used in this disclosure, is a process that automatedly uses training data 204 to generate an algorithm that will be performed by a computing device/module to produce outputs 208 given data provided as inputs 212; this is in contrast to a non-machine-learning software program where the commands to be executed are determined in advance by a user and written in a programming language.

Still referring to FIG. 2, “training data,” as used herein, is data containing correlations that a machine-learning process may use to model relationships between two or more categories of data elements. For instance, and without limitation, training data 204 may include user training set, affliction training set, severity training set, among a plurality of other data entries, each entry representing a set of data elements that were recorded, received, and/or generated together; data elements may be correlated by shared existence in a given data entry, by proximity in a given data entry, or the like. Multiple data entries in training data 204 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 204 according to various correlations; correlations may indicate causative and/or predictive links between categories of data elements, which may be modeled as relationships such as mathematical relationships by machine-learning processes as described in further detail below. Training data 204 may be formatted and/or organized by categories of data elements, for instance by associating data elements with one or more descriptors corresponding to categories of data elements. As a non-limiting example, training data 204 may include data entered in standardized forms by persons or processes, such that entry of a given data element in a given field in a form may be mapped to one or more descriptors of categories. Elements in training data 204 may be linked to descriptors of categories by tags, tokens, or other data elements; for instance, and without limitation, training data 204 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 2, training data 204 may include one or more elements that are not categorized; that is, training data 204 may not be formatted or contain descriptors for some elements of data. Machine-learning algorithms and/or other processes may sort training data 204 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns updated by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 204 to be made applicable for two or more distinct machine-learning algorithms as described in further detail below. Training data 204 used by machine-learning module 200 may correlate any input data as described in this disclosure to any output data as described in this disclosure. As a non-limiting illustrative example, a monitoring set input to monitoring machine-learning model to output the monitoring device.

Further referring to FIG. 2, training data may be filtered, sorted, and/or selected using one or more supervised and/or unsupervised machine-learning processes and/or models as described in further detail below; such models may include without limitation a training data classifier 216. Training data classifier 216 may include a “classifier,” which as used in this disclosure is a machine-learning model as defined below, such as a mathematical model, neural net, or program generated by a machine-learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 200 may generate a classifier using a classification algorithm, defined as a process whereby a computing device and/or any module and/or component operating thereon derives a classifier from training data 204. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 216 may classify elements of training data to specific monitoring devices, or monitoring device types where a monitoring device type may be classified based on the detection method used.

Still referring to FIG. 2, machine-learning module 200 may be configured to perform a lazy-learning process 220 and/or protocol, which may alternatively be referred to as a “lazy loading” or “call-when-needed” process and/or protocol, may be a process whereby machine-learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 204. Heuristic may include selecting some number of highest-ranking associations and/or training data 204 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 2, machine-learning processes as described in this disclosure may be used to generate machine-learning models 224. A “machine-learning model,” as used in this disclosure, is a mathematical and/or algorithmic representation of a relationship between inputs and outputs, as generated using any machine-learning process including without limitation any process as described above, and stored in memory; an input is submitted to a machine-learning model 224 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 224 may be generated by creating an artificial neural network, such as a convolutional neural network comprising an input layer of nodes, one or more intermediate layers, and an output layer of nodes. Connections between nodes may be created via the process of “training” the network, in which elements from a training data 204 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 2, machine-learning algorithms may include at least a supervised machine-learning process 228. At least a supervised machine-learning process 228, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include monitoring training set as described above as inputs, monitoring devices as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 204. Persons skilled in the art, upon reviewing the entirety of this disclosure, will be aware of various possible variations of at least a supervised machine-learning process 228 that may be used to determine relation between inputs and outputs. Supervised machine-learning processes may include classification algorithms as defined above.

Further referring to FIG. 2, machine-learning processes may include at least an unsupervised machine-learning processes 232. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 2, machine-learning module 200 may be designed and configured to create a machine-learning model 224 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be updated to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 2, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

Still referring to FIG. 2, models may be generated using alternative or additional artificial intelligence methods, including without limitation by creating an artificial neural network, such as a convolutional neural network comprising an input layer of nodes, one or more intermediate layers, and an output layer of nodes. Connections between nodes may be created via the process of “training” the network, in which elements from a training data 204 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. This network may be trained using training data 204.

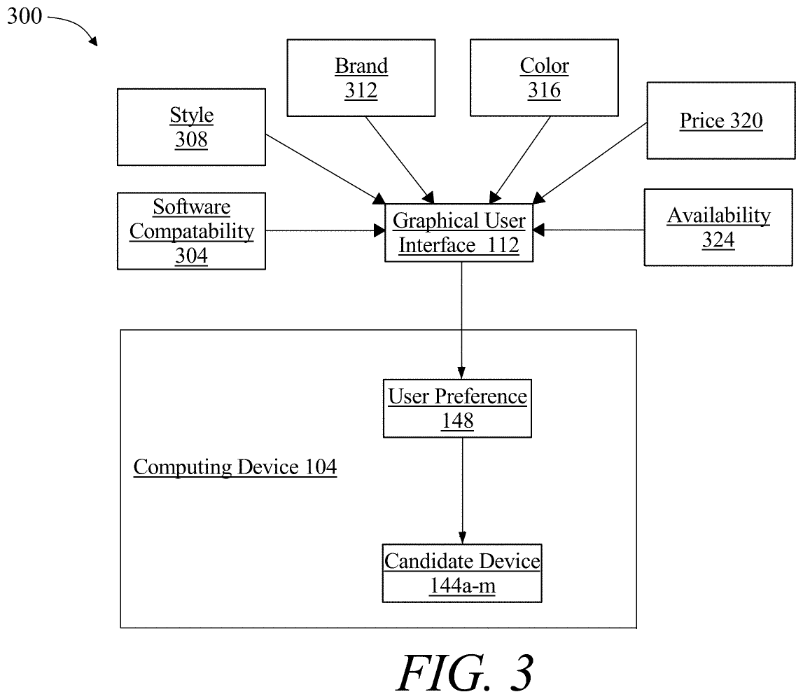

Now referring to FIG. 3 an exemplary embodiment of 300 of user preference 148 inputs according to an embodiment of the invention is illustrated. User preference inputs may be entered at graphical user interface 112 and received by computing device 104. User preference inputs may consist of inputs related to software compatibility 304. As used in this disclosure “software computability” consist of software components or systems which can operate satisfactorily together on the same computing and/or monitoring device, or on different computing and/or monitoring devices linked by a computer network. Software compatibility 304 may include compatibility with one or more monitoring devices on one computing and/or monitoring device yet may include incompatibility with a separate computing and/or monitoring device. User preference inputs may be comprised of a style 308. As used in this disclosure “style” relates to any particular, distinctive, or characteristic form, appearance, character, mode, or display. For example, style 308 may include the distinction of a circular smartwatch as opposed to a square smartwatch. User preference inputs may be composed of a brand 312. As used in this disclosure “brand” may include a type of product manufactured by a particular company under a particular name. Brand 312 may include any unique component of the monitoring device, such as logos, trademarks, or company names relating to the wearable device. For example, and without limitation, brands may include Garmin, Fitbit, Samsung, Apple, Android Wear, Fossil, Misfit, Motorola, Tile, Under Armor, Withings, Moov, iHealth, Polar, Striiv, Huawei, MyKronoz, and the like thereof. User preference inputs may include a color 316. As used in this disclosure “color” is comprised of a characteristic of visual perception. Color 316 may include all color categories such as red, orange, yellow, green, blue, or purple and all other color variances of the 10,000,000 color variances associated with the color categories. User preference inputs may include a price 320. As used in this disclosure “price” is comprised of the amount of currency that has to be paid to acquire a given monitoring device. Price 320 may include any denomination or currency that is indicative of the monitoring device. Price 320 may be sub-categorized into price ranges, wherein a price range sets a specific lower and upper limit as to the amount of currency the user would like to spend on a monitoring device. User preference inputs may include an availability 324. As used in this disclosure “availability” is comprised of the accessibility of a monitoring device to the user such that the user may purchase or obtain the monitoring device.

With continued reference to FIG. 3, computing device 104 may receive an availability 324 relating to a user based on a user location. Computing device 104 may receive an element of user geolocation. An “element of user geolocation,” as used in this disclosure, is an identification of a real-world geographical location of a user. An element of user geolocation may be obtained from a radar source, remote device such as a mobile phone, and/or internet connected device location. An element of user geolocation may include a global positioning system (GPS) of a user. An element of user geolocation may include geographic coordinates that may specify the latitude and longitude of a particular location where a user is located. Computing device 104 may utilize an element of user geolocation to locate a monitoring device located within the user geolocation. In an embodiment, a user may specify that the user only seeks to obtain a monitoring device within a ten-mile radius of the user.

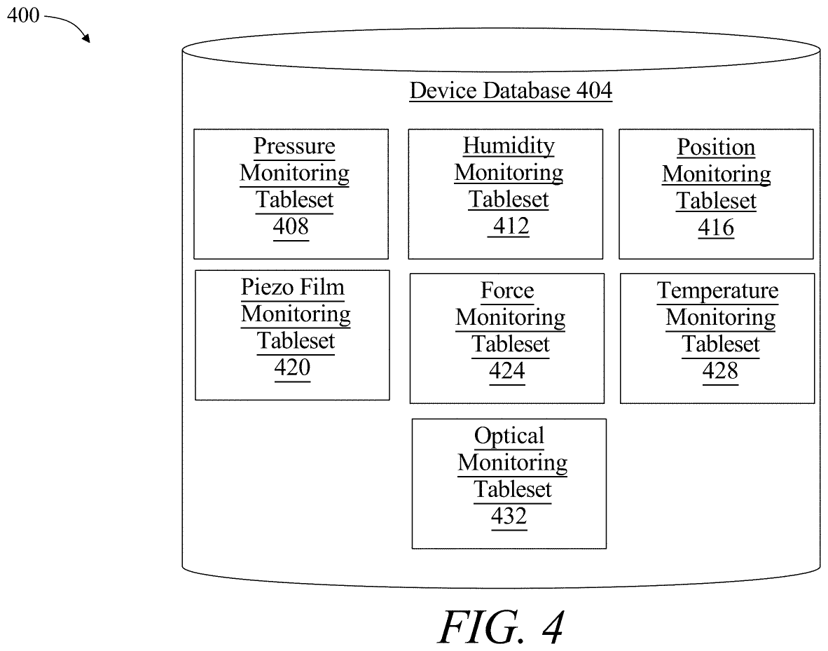

Now referring to FIG. 4, an exemplary embodiment 400 of a device database 404 is illustrated. Device database 404 may be implemented, without limitation, as a relational database, a key-value retrieval database such as a NOSQL database, or any other format or structure for use as a database that a person skilled in the art would recognize as suitable upon review of the entirety of this disclosure. Database may alternatively or additionally be implemented using a distributed data storage protocol and/or data structure, such as a distributed hash table or the like. Database may include a plurality of data entries and/or records as described above. Data entries in a database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistently with this disclosure. Device database may 404 may include one or more tables, including without limitation, a pressure monitoring table set 408; pressure monitoring table set may include devices capable of monitoring pressure information concerning a human subject, including without limitation swelling, blood pressure, eye pressure, bladder pressure, brain and/or spinal fluid pressure, digestive tract pressure, or the like thereof. Device database 404 may include a humidity monitoring table set 412, which may contain monitoring devices capable of monitoring a humidity information concerning human subject, including without limitation inhalation humidity, exhalation humidity, evaporative cooling, or the like thereof. Device database 404 may include a position monitoring table set 416, which may contain monitoring devices capable of monitoring position information concerning a human subject, including without limitation geolocation, cardinal direction, speed, and the like thereof. Device database 404 may include a piezo film monitoring table set 420, which may contain monitoring devices capable of monitoring using piezo electric sensors, wherein a human subject may be monitored for impact location, physical activity, limb activity, rapid eye movement, and the like thereof. Device database 404 may include a force monitoring table set 424, which may contain monitoring devices capable of monitoring a force associated with dynamic and static loads may be monitored associated with limb movement, prosthetics, muscular contraction, and the like thereof. Device database 404 may include a temperature monitoring table set 428, which may contain monitoring devices capable of monitoring temperature of a human subject, wherein a human subject may be monitored for occipital temperature, dermal temperature, rectal temperature, axillary temperature, and the like thereof. Device database 404 may include an optical monitoring table set 432, which may contain monitoring devices capable of monitoring a human subject using optical sensors, wherein optical sensors may utilize infrared, x-ray absorptiometry, bioimpedance spectroscopy, visible light spectroscopy, and the like thereof.

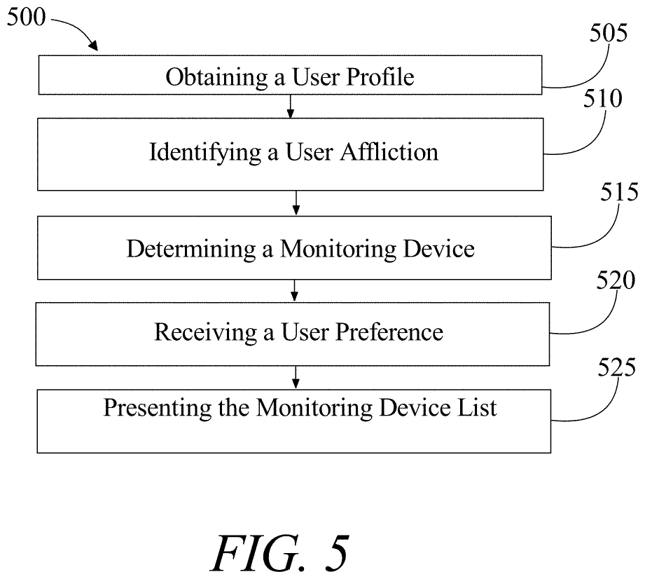

Now referring to FIG. 5, an exemplary embodiment of a method 500 of presenting a monitoring device identification. At step 505 a computing device 104 obtains a user profile 108 from a graphical user interface 112. User profile 108 includes any of the user profile 108 as described above in reference to FIGS. 1-3. User profile 108 may include any quality, element, or characteristic regarding a user's health and wellness state. For instance, and without limitation, user profile 108 may include a biological extraction. For instance, and without limitation, user profile 108 may include a questionnaire and/or survey.

Still referring to FIG. 5, at step 510 a computing device identifies a user condition 116 as a function of user profile 108. User condition 116 includes any of the user condition 116 as described above in reference to FIGS. 1-3. User condition may include any physical, spiritual, or psychological condition that is affecting the user. For instance and without limitation, user impact may include COVID-19, chakra blockage, and/or Alzheimer's disease. Computing device 104 may identify user condition 104 as a function of one or more machine-learning processes as described in FIGS. 1-3. Computing device 104 may identify user condition 116 as a function of a condition machine learning process 120. Condition machine-learning process includes any of the condition machine-learning process 120 described above in reference to FIGS. 1-3. For instance, and without limitation, condition machine-learning process 120 may include a supervised machine-learning process or an unsupervised machine-learning process. Condition machine learning process 120 may include a classification process, such as for example naïve Bayes, k-nearest neighbor, decision tree, and/or random forest. Classification processes include any of the classification processes as described above in reference to FIGS. 1-3. Condition machine-learning process 120 may be configured using a condition training set 124. Condition training set 124 includes any of the condition training set 124 as described above in reference to FIGS. 1-3. Condition training set may include, without limitation, questionnaires, surveys, and/or biological extractions that correlate to a condition that a user may be experiencing. For example, and without limitation a condition training set may relate a presence of a specific biomarker, such as glucose, with a particular condition, such as diabetes.

Still referring to FIG. 5, at step 515 a computing device determines a monitoring device 128a of a plurality of monitoring devices 128a-m relating to the user condition. Monitoring device 128 includes any of the monitoring device 128 as described above in reference to FIGS. 1-3. For instance monitoring device 128 may include a device or fabric that a user maintains in close proximity to the user, such as a clothing, jewelry, or accessory such that the device can inform a user about user condition 116. Monitoring device 128a-m may be determined as a function of measuring situational information. Situational information includes any of the situational information as described above in reference to FIGS. 1-3. Situational information relates the location of an at least first condition element to the location of an at least second condition element. Condition element includes any of the condition element as described above in reference to FIGS. 1-3. Situational information may relate one or more locations of one or more condition elements to determine one or more monitoring devices that at least monitor a user condition. For example, and without limitation, two condition elements of dopamine concentration and catecholamine concentration may be necessary to monitor the user condition of depression, wherein the two condition elements are monitored using one or more monitoring devices located at one or more locations of the body.

Computing device 104 identifies monitoring device 128 as a function of one or more machine-learning processes as described in FIGS. 1-3. Computing device 104 identifies monitoring device 128 as a function of a monitoring machine learning process 132. Monitoring machine-learning process 132 includes any of the monitoring machine-learning process 132 described above in reference to FIGS. 1-3. For instance, and without limitation, monitoring machine-learning process 132 may include a supervised machine-learning process or an unsupervised machine-learning process. Monitoring machine-learning process 132 may include a classification process, such as for example naïve Bayes, k-nearest neighbor, decision tree, and/or random forest. Classification processes include any of the classification processes as described above in reference to FIGS. 1-3. Monitoring machine-learning process 132 is configured using a monitoring training set 136. Monitoring training set 136 includes any of the monitoring training set 136 as described above in reference to FIGS. 1-3. For example, and without limitation a monitoring training set may include a condition element correlated to a detection method, wherein a condition element is a quality, component, and/or integrant, such as a cough or cognitive dysfunction and a detection method is any route, technique, and/or strategy for monitoring the user condition. Computing device 104 determines the monitoring device as a function of the monitoring machine-learning process and the user condition. Computing device 104 may configure monitoring machine-learning process 132 to determine a device score, wherein each monitoring device receives a generated score associated with the user condition. Computing device 104 may utilize a classifier 140. Classifier 140 includes any of the classifier 140 as described above in reference to FIGS. 1-3. Classifier 140 may include, without limitation, receiving a user condition input and outputting one or more monitoring devices that at least detect one or more elements of the user condition, such as heart rate, blood pressure, or the like thereof.

Still referring to FIG. 5, at step 520, computing device 104 may receive a user preference 148 from the displayed plurality of candidate monitoring devices 144a-m. User preference 148 includes any of the user preference 148 as described above in reference to FIGS. 1-3. For instance, and without limitation, user preference may include wish, desire, want, and/or urge regarding a style, color, brand, software, price, availability, and the like thereof of the monitoring device. Computing device 104 may then select the monitoring device as a function of the user preference and user condition. Computing device 104 may generate a loss function utilizing ranked plurality of monitoring devices as variables and user conditions. Computing device 104 may assigned a weighted variable score to a ranked monitoring device. Computing device 104 may minimize the loss function utilizing any of the methodologies as described above in reference to FIGS. 1-3. Computing device 104 generates a loss function utilizing ranked plurality of monitoring devices and user conditions to calculate a difference between the ranked plurality of monitoring devices and user conditions as a function of minimizing the loss function. Computing device 104 determines whether monitoring device is capable of monitoring a user condition as a function of minimizing a loss function.

Still referring to FIG. 5, at step 525, a computing device presents monitoring device 128 at the graphical user interface 112. For instance, and without limitation, computing device 104 may present monitoring device 128 at graphical user interface, such that monitoring device 128 fulfills user preference 148 requirements as well as the condition 116 requirements. Computing device 104 may present one or more monitoring devices such that the user condition may be monitored as required. For instance, and without limitation, one monitoring device may be presented for the monitoring of the user condition associated with obesity, through the detection method of heart rate. As a further non-limiting example, multiple monitoring devices may be presented for the monitoring of a user condition associated with influenza, such as a heart monitor device as well as a sinus cavity monitoring device.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

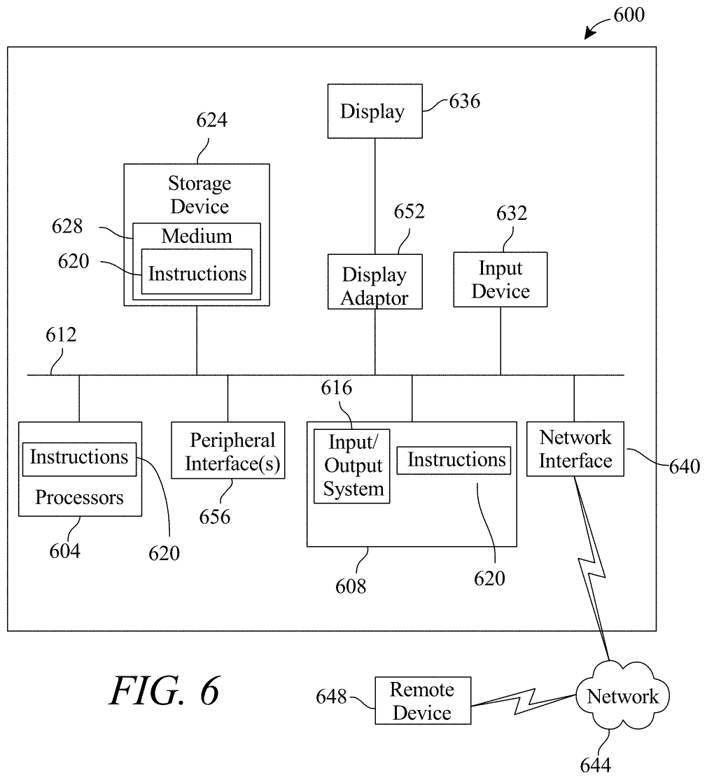

FIG. 6 shows a diagrammatic representation of one embodiment of a computing device in the exemplary form of a computer system 600 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 600 includes a processor 604 and a memory 608 that communicate with each other, and with other components, via a bus 612. Bus 612 may include any of several types of bus structures including, but not limited to, a memory bus, a memory controller, a peripheral bus, a local bus, and any combinations thereof, using any of a variety of bus architectures.

Processor 604 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 604 may be organized according to Von Neumann and/or Harvard architecture as a non-limiting example. Processor 604 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC)

Memory 608 may include various components (e.g., machine-readable media) including, but not limited to, a random-access memory component, a read only component, and any combinations thereof. In one example, a basic input/output system 616 (BIOS), including basic routines that help to transfer information between elements within computer system 600, such as during start-up, may be stored in memory 608. Memory 608 may also include (e.g., stored on one or more machine-readable media) instructions (e.g., software) 620 embodying any one or more of the aspects and/or methodologies of the present disclosure. In another example, memory 608 may further include any number of program modules including, but not limited to, an operating system, one or more application programs, other program modules, program data, and any combinations thereof.

Computer system 600 may also include a storage device 624. Examples of a storage device (e.g., storage device 624) include, but are not limited to, a hard disk drive, a magnetic disk drive, an optical disc drive in combination with an optical medium, a solid-state memory device, and any combinations thereof. Storage device 624 may be connected to bus 612 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 624 (or one or more components thereof) may be removably interfaced with computer system 600 (e.g., via an external port connector (not shown)). Particularly, storage device 624 and an associated machine-readable medium 628 may provide nonvolatile and/or volatile storage of machine-readable instructions, data structures, program modules, and/or other data for computer system 600. In one example, software 620 may reside, completely or partially, within machine-readable medium 628. In another example, software 620 may reside, completely or partially, within processor 604.

Computer system 600 may also include an input device 632. In one example, a user of computer system 600 may enter commands and/or other information into computer system 600 via input device 632. Examples of an input device 632 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 632 may be interfaced to bus 612 via any of a variety of interfaces (not shown) including, but not limited to, a serial interface, a parallel interface, a game port, a USB interface, a FIREWIRE interface, a direct interface to bus 612, and any combinations thereof. Input device 632 may include a touch screen interface that may be a part of or separate from display 636, discussed further below. Input device 632 may be utilized as a user selection device for selecting one or more graphical representations in a graphical interface as described above.

A user may also input commands and/or other information to computer system 600 via storage device 624 (e.g., a removable disk drive, a flash drive, etc.) and/or network interface device 640. A network interface device, such as network interface device 640, may be utilized for connecting computer system 600 to one or more of a variety of networks, such as network 644, and one or more remote devices 648 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 644, may employ a wired and/or a wireless mode of communication. In general, any network topology may be used. Information (e.g., data, software 620, etc.) may be communicated to and/or from computer system 600 via network interface device 640.

Computer system 600 may further include a video display adapter 652 for communicating a displayable image to a display device, such as display device 636. Examples of a display device include, but are not limited to, a liquid crystal display (LCD), a cathode ray tube (CRT), a plasma display, a light emitting diode (LED) display, and any combinations thereof. Display adapter 652 and display device 636 may be utilized in combination with processor 604 to provide graphical representations of aspects of the present disclosure. In addition to a display device, computer system 600 may include one or more other peripheral output devices including, but not limited to, an audio speaker, a printer, and any combinations thereof. Such peripheral output devices may be connected to bus 612 via a peripheral interface 656. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve systems and methods according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for presenting a monitoring device identification, the system comprising:
a computing device, the computing device configured to:
obtain, from a graphical user interface, a user profile;
identify a user condition as a function of the user profile;
determine, as a function of the user condition, a monitoring device of a plurality of monitoring devices relating to the user condition, wherein determining further comprises:
obtaining a monitor training set, wherein the monitor training set relates a condition element to a detection method; and
determining the monitoring device as a function of a monitoring machine-learning process and the user condition, wherein the monitoring machine process is configured as a function of the monitoring training set; and
present the monitoring device at the graphical user interface.

2. The system of claim 1, wherein the user profile further comprises a biological extraction.

3. The system of claim 1, wherein identifying the user condition further comprises:
obtaining a condition training set relating at least a user profile to a condition; and
identifying the user condition, as a function of the condition training set, using a condition machine-learning process, wherein the condition machine learning process is configured using the condition training set.

4. The system of claim 1, wherein the detection method includes a method to indicate a condition state.

5. The system of claim 1, wherein determining the monitoring device further comprises measuring situational information, wherein as a function of measuring the situational information a first condition element is monitored in conjunction with a second condition element.

6. The system of claim 5, wherein situational information further comprises the location of the first condition element in relation to the second condition element.

7. The system of claim 1, wherein the computing device is configured to perform the monitoring machine-learning process by determining a device enumeration.

8. The system of claim 1, wherein the computing device is configured to generate the monitoring machine-learning process by determining a plurality of candidate monitoring devices; and
selecting the monitoring device from the plurality of candidate devices.

9. The system of claim 8, wherein determining the monitoring device further comprises:
presenting on the computing device a plurality of candidate monitoring devices;
obtaining a user preference;
ranking the plurality of candidate monitoring devices as a function of the user preference; and
selecting the monitoring device as a function of the user preference.

10. The system of claim 9 further comprising:
generating a parameter estimation using the ranked plurality of candidate monitoring devices and the user condition;
computing a difference between the ranked plurality of candidate monitoring devices and the user condition as a function of the parameter estimation; and
selecting the monitoring device for the user as a function of computing the difference.

11. A method for presenting a monitoring device identification, the method comprising:
obtaining, by a computing device, from a graphical user interface, a user profile;
identifying, by the computing device, a user condition as a function of the user profile;
determining, by the computing device, as a function of the user condition, a monitoring device of a plurality of monitoring devices relating to the user condition; wherein determining further comprises:
obtaining a monitor training set, wherein the monitor training set relates a condition element to a detection method; and
determining the monitoring device as a function of a monitoring machine-learning process and the user condition, wherein the monitoring machine process is configured as a function of the monitoring training set; and
presenting, by the computing device, the monitoring device at the graphical user interface.

12. The method of claim 11, wherein the user profile further comprises a biological extraction.

13. The method of claim 11, wherein identifying a user condition further comprises:
obtaining a condition training set relating at least a user profile to a condition; and
identifying the user condition, as a function of the condition training set, using a condition machine-learning process; the condition machine learning process is configured using the condition training set.

14. The method of claim 11, wherein the detection method includes a method to indicate a condition state.

15. The method of claim 11, wherein determining the monitoring device further comprises measuring situational information, wherein as a function of measuring the situational information, a first condition element is monitored in conjunction with a second condition element.

16. The method of claim 15, wherein situational information further comprises the location of the first condition element in relation to the second condition element.

17. The method of claim 11, wherein the computing device is configured to perform the monitoring machine-learning process by determining a device enumeration.

18. The method of claim 11, wherein the computing device is configured to generate the monitoring machine-learning process by determining a plurality of candidate monitoring devices; and
selecting the monitoring device from the plurality of candidate devices.

19. The method of claim 18, wherein determining the monitoring device further comprises:
presenting on the computing device a plurality of candidate monitoring devices;
obtaining a user preference;
ranking the plurality of candidate monitoring devices as a function of the user preference; and
selecting the monitoring device as a function of the user preference.

20. The method of claim 19 further comprising:
generating a parameter estimation using the ranked plurality of candidate monitoring devices and the user condition;
computing a difference between the ranked plurality of candidate monitoring devices and the user condition as a function of the parameter estimation; and
selecting the monitoring device for the user as a function of computing the difference.
